(19) United States
(12) Patent Application Publication
(10) Pub. No.: US 2023/0024180 A1
(43) Pub. Date: Jan. 26, 2023

(54) SYSTEMS AND METHODS FOR TRACKING MEDICAL INSTRUMENTS CLEANING AND STERILIZATION

(71) Applicant: SciCan Ltd., Toronto, CA (US)

(72) Inventors: Martin SCHAUFELBERGER, Altstätten (CH); Gabriel NEAGOE, Richmond Hill (CA); Dorin CIORACA, Thornhill (CA)

(21) Appl. No.: 17/742,996

(22) Filed: May 12, 2022

Related U.S. Application Data
(60) Provisional application No. 63/188,895, filed on May 14, 2021.

(51) Int. Cl. G01N 31/22; A61L 2/28; A61L 2/16

(52) U.S. Cl. CPC G01N 31/226; A61L 2/28; A61L 2/16; A61L 2202/24

(57) ABSTRACT

Methods and systems for tracking a cassette containing one or more instruments in an instrument reprocessing are provided. A method for tracking an cassette housing one or more instruments in an instrument reprocessing, comprising: sterilizing, at a sterilizer, the cassette; receiving, at a server, a chemical indicator identification encrypted by a private key after the cassette is sterilized; decrypting, at the server, the chemical indicator identification with a corresponding public key; and in response to decryption of the chemical indicator identification, authenticating, at the server, the chemical indicator identification.

### CROSS-REFERENCE TO RELATED APPLICATION

This application claims the benefits of priority to U.S. Provisional Patent Application No. 63/188,895, filed May 14, 2021, titled SYSTEMS AND METHODS FOR TRACKING MEDICAL INSTRUMENTS CLEANING AND STERILIZATION, the contents of which are hereby expressly incorporated into the present application by reference in their entirety.

### FIELD

The present application relates to medical instrument reprocessing, in particular to systems and methods for tracking medical instruments reprocessing.

### BACKGROUND

A typical medical instrument reprocessing system consists of ultrasonic washers, washers or washer disinfectors and sterilization equipment. Once medical instruments, such as dental or surgical instruments, were used for procedures, personnel use the reprocessing system to wash and sterilize the medical instruments to allow reuse.

To record the completion of medical instrument reprocessing steps, the personnel may manually record or track, such as on a paper or electronic log, the steps performed on a medical instrument and reprocessing states of the medical instrument, including the status of the class 4 and 5 chemical indicators, to record successful completion of the sterilization cycle and sterile status of the medical instrument.

However, manual recording or tracking of medical instrument reprocessing steps is prone to human errors, and may be inaccurate due to missing or inaccurate information. As well, the records of reprocessing a medical instrument may also be tampered.

### SUMMARY

The present application provides a tracking system for medical instrument reprocessing. The system allows batch or wrapped medical instruments tracking across all infection control steps, including washing, disinfection, and sterilization.

The systems and methods of the present application may use RFID and barcode for medical instrument tracking, and use traceable chemical indicator for sterilization that allows machine readable status of sterilization.

As well, the systems and methods use asymmetric encryption and blockchain to authenticate and file successful sterilization cycles with trusted third parties.

In an aspect, there is provided a method for tracking an cassette housing one or more instruments in an instrument reprocessing, comprising: sterilizing, at a sterilizer, the cassette; receiving, at a server, a chemical indicator identification encrypted by a private key after the cassette is sterilized; decrypting, at the server, the chemical indicator identification with a corresponding public key; and in response to decryption of the chemical indicator identification, authenticating, at the server, the chemical indicator identification.

### BRIEF DESCRIPTION OF THE DRAWINGS

Reference will now be made, by way of example, to the accompanying drawings which show example embodiments of the present application, and in which:

FIG. 1 is a diagram illustrating a tracking process of medical instrument reprocessing, according to an embodiment of the present application;

FIG. 2 is a diagram illustrating reading of cassettes ID in the tracking process of medical instrument reprocessing in FIG. 1;

FIG. 3 is a diagram illustrating an example of a chemical indicator prior to and after a successful sterilization step;

FIG. 4A is a diagram illustrating the steps of chemical indicator ID authentication and validation, and FIG. 4B is a diagram illustrating a 2D code representation of the Encrypted Class 5 indicator information;

FIG. 5 is a diagram illustrating new chemical indicator ID generation;

FIG. 6 is a diagram illustrate the chemical indicator ID status change;

FIG. 7A is a diagram illustrating chemical indicator information storage in an exemplary blockchain, according to an embodiment;

FIG. 7B is a diagram illustrating an exemplary blockchain structure and values of the blockchain in FIG. 7A; and

FIG. 8 is a diagram illustrating an instrument traceability data model, according to an embodiment.

Similar reference numerals may have been used in different figures to denote similar components.

### DESCRIPTION OF EXAMPLE EMBODIMENTS

The tracking systems and methods of the present application provide capability of tracking of all the steps of reprocessing medical instruments. The tracking systems and methods substantially or entirely eliminate potential error due to user assessment of the chemical indicator, substantially increase accuracy of records or documentation for tracking reprocessing medical instruments as each chemical indicator used in the reprocessing has a unique identification (ID) authenticated via a trusted third party. Finally, records produced at each step of medical instrument reprocessing are securely stored in a blockchain. Each block has a time stamp and the blockchain makes the records safe against modifications.

It is important or even mandatory in some regulatory domains to keep records or documentation accurately tracking each steps of infection reprocessing of a medical instrument. The instrument reprocessing system may be used to (1) clean and sterilize the instruments for reuse and (2) to provide evidence to support successful completion of the reprocessing activity.

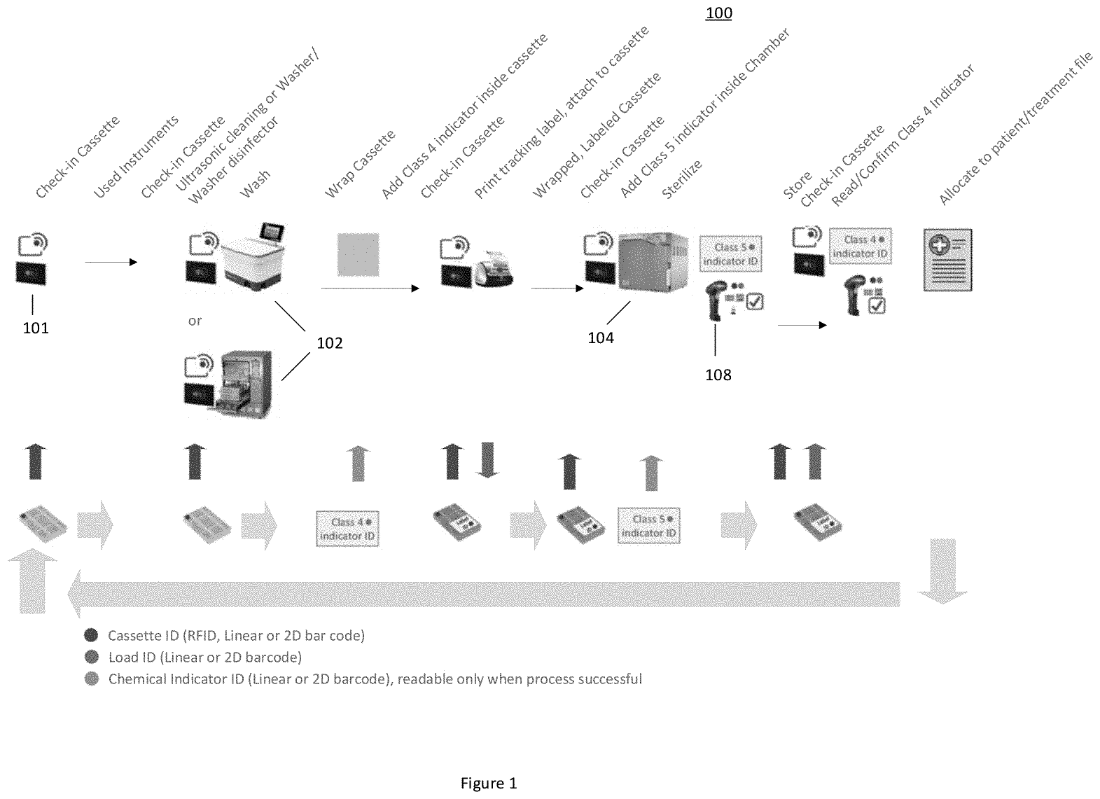

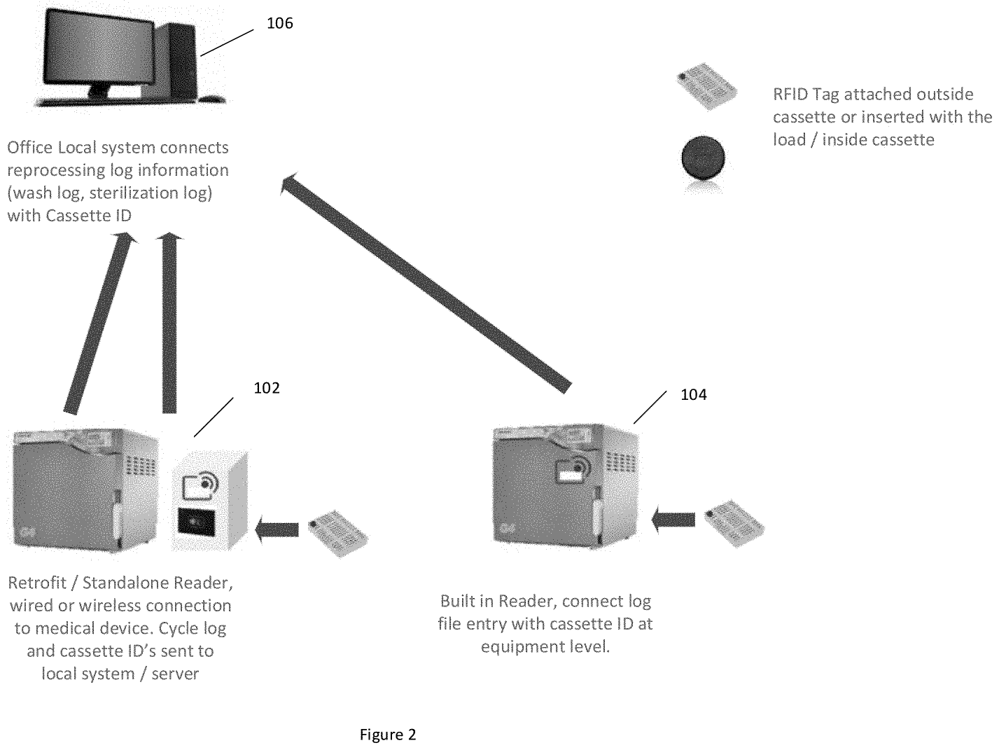

As illustrated in FIG. 1, an instrument reprocessing system 100 may include one or more ultrasonic washers, washers, or washer disinfectors 102 and one or more sterilization equipment 104. As illustrated in FIG. 2, the instrument reprocessing system 100 may also include a local computer or server 106, which may be a cloud server. The instruments may be any instruments that needs to be washed and/or sterilized, including medical instruments for human or animals.

After an instrument is used, the instrument reprocessing system 100 may be used to wash and sterilize the instrument. One or more used instruments may be placed in a cassette 101 for cleaning and sterilizing. As illustrated in the example of FIG. 1, once instruments used on a patient and returned for reprocessing, one or more used instruments may be placed in a cassette 101. The cassette 101 has a unique cassette identification (ID) in the system 100 to uniquely identify the cassette 101. The instruments in the cassette 101 are associate with the cassette ID. The cassette ID may be an RFID, a linear or 2D barcode. An RFID may be attached to an external surface, an internal surface of the cassette 101, or inserted with the load of the instruments in the cassette 101.

The washer 102 may be used to wash used instruments in the cassette 101, for example by ultrasonic cleaning or washing. Before the cassette 101 is loaded into the washer 102, the cassette ID is checked in the server 106. As illustrated in FIG. 2, in the check in process, the cassette ID may be read in the local computer 106 using a reading device 108, for example, by reading the RFID using an RFID reader, or scanning the barcode using a barcode scanning device. The reading device 108 may be a retrofit or standalone device or a device integrated with the computer 106. After the reading device 108 has read the cassette ID, the reading device 108 may communicate the cassette ID to the computer 106. Checking the cassette 101 in the computer 106 indicates that the instruments in the cassette 101 are to be washed by the washer 102. As such, the local computer 106 may link the cassette 101 with the cassette ID to a specific wash or sterilization (to be described below) cycle and a specific washer 102 to allow traceability.

In the example of FIG. 1, after the cassette 101 is washed, a class 4 may be added inside the cassette 101. The class 4 indicator is stored inside the cassette 101 and is added prior to the cassette 101 being wrapped. The cassette 101 may then be wrapped. Prior to the instruments in the cassette 101 being used, wrapping cassette 101 allows sterile state of sterilized cassette to be maintained when the cassette 101 is stored. The chemical indicators currently used require user training as a pass/fail criterion requires manual visual assessment by the user and errors may be incurred in the assessment.

A class 4 chemical indicator is accessible to the user once the wrap of the cassette 101 is removed prior to instruments being used.

In some examples, the wrapped cassette 101 may be checked into the computer 106 or sterilizer 104 with the cassette ID. In response, the computer 106 may generate a tracking label with an load ID. A tracking label displays human and machine readable information, such as the load ID, to create options for information access during integration with a patient/treatment file. Load ID tracks the cassette 101 throughout the sterilization reprocessing session. The load ID of the tracking label may be associated with the cassette ID for a sterilization reprocessing session. Generation of the tracking label indicates that the instruments in the cassette 101 have been washed and that a class 4 chemical indicator is attached to the cassette 101. The tracking label with an load ID may be attached to the wrapped cassette 101. The wrapped cassette 101 may be checked in the computer 106 by scanning the load ID on the tracking label before the cassette 101 is sterilized in the sterilization equipment 104.

Next, the wrapped cassette 101 is placed within the chamber of the sterilizer 104. As illustrated in FIG. 1, in some examples, a class 5 chemical indicator may be added to the chamber of sterilizer 104 prior to starting the sterilization cycle. The Class 5 indicator is loaded inside the sterilization chamber outside the cassette 101. A class 5 indicator is typically used as an extra monitoring control to allow release of the cassette 101 in the chamber at the end of the sterilization cycle.

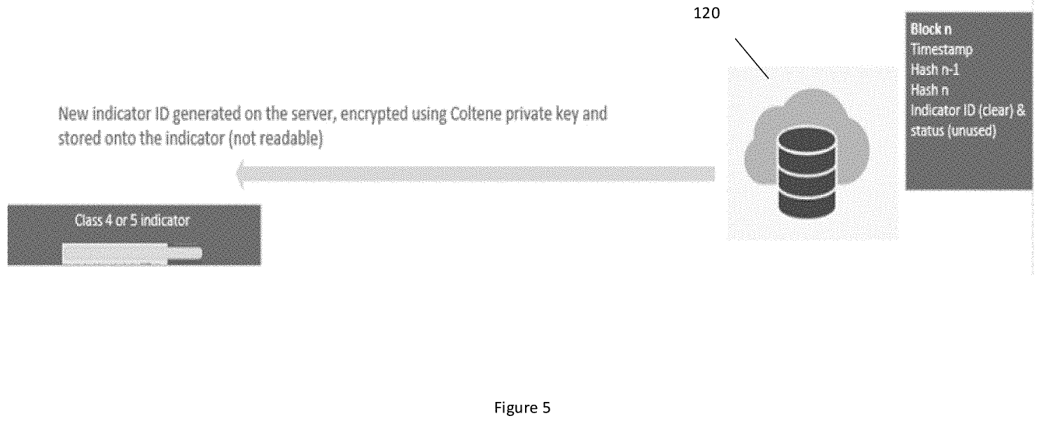

Each chemical indicator 4 and 5 has a unique identification (ID) authenticated via a trusted third party, for example at the server 120. As illustrated in FIG. 5 and Table 1 below, a class 4 or 5 chemical indicator ID may be encrypted with a private key, such as a Coltene private key. For example, the private key may be in PKCS#8 format, as set out in the example in Table 1 below. The encrypted class 4 or 5 chemical indicator ID may be a one dimensional or two dimensional scrambled code which is invisible prior to the sterilization. The encrypted new class 4 or 5 indicator ID may be stored onto the indicator in a concealed manner and is not readable by a reading device 108.

At the end of the sterilization cycle, the sterilizer 104 provides an indication whether the sterilization process ended successfully based on the measurements of internal temperature and pressure sensors of the sterilizer 104. The class 4 indicator and class 5 indicator changes state and become readable when the sterilization cycle met the required sterilization parameters, such as internal temperature and pressure. The class 4 indicator and class 5 chemical indicator functions fully independent of the machine control and sensors of the sterilizer 104 and provides an additional confirmation when the sterilization parameters have been satisfied as measured by the class 4 indicator and class 5 indicator.

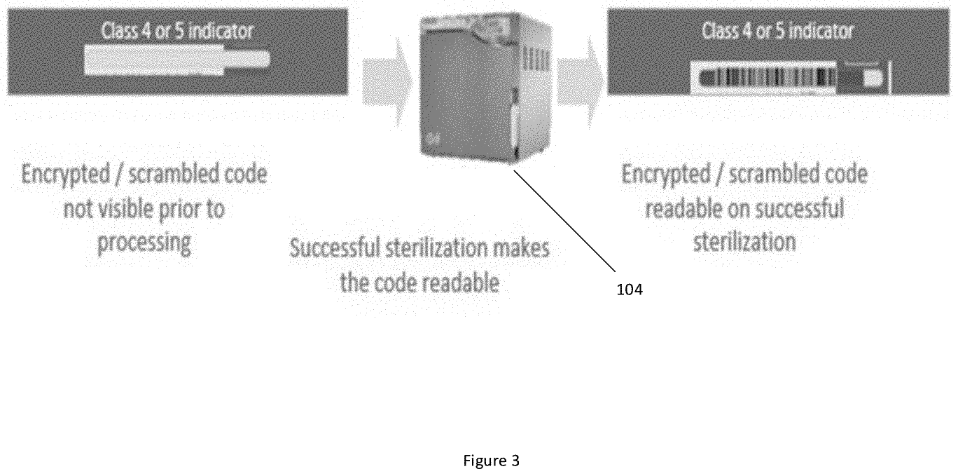

As illustrated in FIG. 3, the class 4 or 5 chemical indicator scrambled ID is not readable for before a successful sterilization. A successful sterilization of the instruments in the cassette 101 in the sterilization equipment 104 reveals to the class 4 or 5 chemical indicator scramble code. The revealed class 5 indicator in the chamber allows a quick release of the cassette 101. Once sterilization is completed, the class 5 indicator may be removed from the sterilizer chamber.

A scanning device 108 may scan the revealed code of class 5 chemical indicator and check in the cassette 101 in the computer 106 or sterilizer 104 with scanned class 5 chemical indicator code. The class 5 chemical indicator code in the local computer 106 or sterilizer 104 indicates that the instruments in the cassette 101 are successfully sterilized.

As the class 4 chemical indicator is placed inside the wrapped cassette 101, the result for the cassette 101 is available only when the cassette 101 is unwrapped prior to using the instruments in the cassette 101.

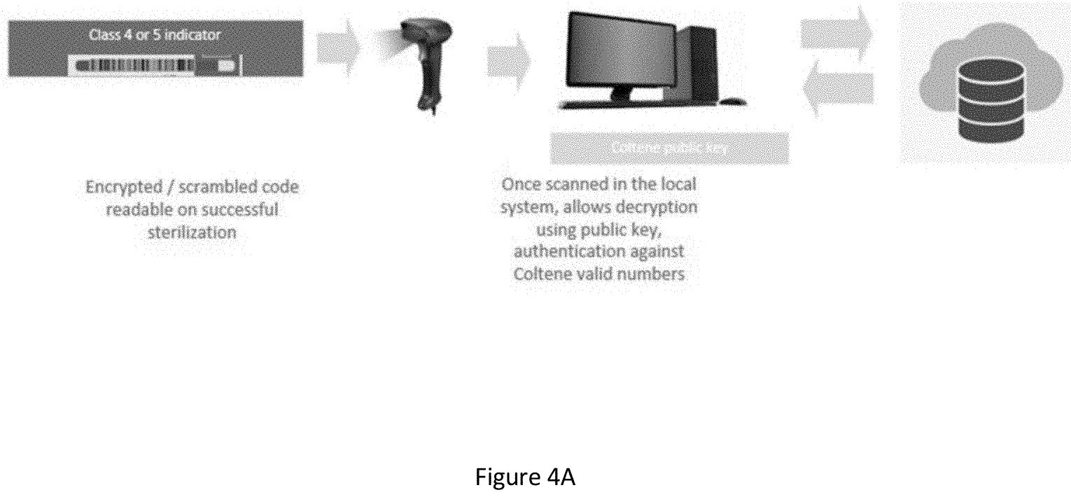

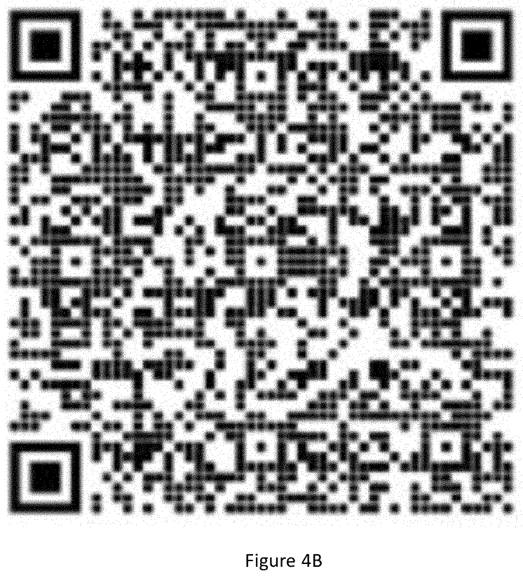

As illustrated in the example of FIG. 4A, the revealed ID of class 4 or 5 indicator can be scanned into the computer 106, and be validated of being an authentic ID at the computer 106 by decrypting the revealed class 4 or 5 indicator ID using a public key, such as a Coltene public key. If the local computer 106 can decrypt the class 4 or 5 indicator ID using a public key, the class 4 and 5 indicator ID is an authentic ID. As set out in the example in Table 1 below, the public key may be in format. The table below shows an example of encryption and decryption of a class 5 indicator ID using private key and public key:

TABLE 1

| | |
|---|---|
| Plain text Class 5 Indicator | MFG: Coltene |
| information | TYPE: Class 5 |
| | MFGDATE: YYYYMMDD |
| | BATCH: YYWWBBBBBB |
| | SN: AA000000A00001 |
| | CHK: XXXXXXXXXXXXXXXX |
| Private Key | MIICdwIBADANBgkqhkiG9w0BAQEFAASCAmEwggJd |
| Private key in PKCS#8 format | AgEAAoGBANztjkp + G2gtduClRCIwKPAJIoauLpv84dJ |
| | HhzsAwY8NEnY5uR7i1xwPiTQnwvlnS3dlYJbQXtWH + |
| | Rv2OZL1KOPzb8OIG08IC/HXxaEUROP9kf0K5nEt5YS |
| | bIwOkdK68WqfNjdu6hDfBr8bVeXg491S/NfnN1NI + ji |
| | ukJcHVCQ3pAgMBAAECgYEAtyphIVOiZ2HiQGfksC + bk |
| | T8Rl/XLgNpNY2iYLov27iIZX1mGKJxLWeFrkIkytlI0m |
| | D29sev55DFCkDM5EqUdiy1RdU/oJjkmEM6 + BYLXT/ |
| | JDmPfr6pSn83w0Zw5bK7j6ucsZYFUGJjTAltlXI + TLu |
| | 0Rz5aOqOkThvuysod3awLOCQQD58Qgxl6GjztXuDK |
| | asaaYS/2lwWC0wNQbfN + o999E5ZE2D0lpUOa + 51Co |
| | WqA6cc3wj7L + 7wyS + 4BUT3kvU1E0zAkEA4kh8NOjh |
| | Rfh40MMEZkKSLwYH9O/sZrmxKzze + Vlmv261lqlFFyB |
| | EnTftQ/S6m3dTzDDVvH1iGZOoT + 5wZ + wgcwJAdOBH |
| | SplzVpxD4Xs8/Wax6DRTY9t31hEV04/cE3os9j93F + tnn |
| | fcTuzwBKnMP0g0Wwd3VksGgDznN4WqT30QfjQJBAKu |
| | 7q1hUbbO20apOd2ZMm7lIcH16IQYNY + KWvfqr2QjsMr |
| | B3ItJcUPao3U83yu/2bgiH/46f8fXLVG/lXDXixPUCQDrH |
| | E9Sytfh3PKwciBHXfQSTFU3UKQwU4dlnECNbThw3xCv |
| | lIRJO + /o1DX2JRMjjq/wcYB/3u1tYIVHArjEGfCE = |
| Encrypted Class 5 indicator | RUUQKOTV/ZaxskXjZ8TKruSXhf5OP7Wk8i/jdFtKwsEIIkCq |
| information (Encrypted using the | SrWWRoGgOlrOufEwpM97b6aglEp2TzRNITefNLaVAT6KJT |
| Private Key, | sYnPpS2zDHqn7ur3s2hdmGoKWzc+Yj0v4MqG3YUP+8wlT |
| Cipher: RSA/ECB/PKCSIPadding, | pG2DigOqLsAnwXgd8b5+qU7Qr5eJ5BMo = |
| BASE64 representation) | |
| Encrypted Class 5 indicator | RUUQKOTV/ZaxskXjZ8TKruSXhf5OP7Wk8i/jdFtKwsEIIkCq |
| information | SrWWRoGgOlr0ufEwpM97b6ag1Ep2TzRNITefNLaVAT6KJT |
| (Scanned from 2D code) | sYnPpS2zDHqn7ur3s2hdmGoKWzc + Yj0v4MqG3YUP + 8wlT |
| | pG2DigOqLsAnwXgd8b5 + qU7Qr5eJ5BMo = |
| Public Key | MIGfMA0GCSqGSIb3DQEBAQUAA4GNADCBiQKBgQDc7Y5 |
| Public key is in X.509 format. | KfhtoLXbgpUQiMCjwCSKGri6b/OHSR4c7AMGPDRJ2Obke4 |
| | tccD4k0J8L5Z0t3ZWCW0F7Vh/kb9jmS9Sjj82/DiBtPCAvx18W |
| | hFETj/ZH9CuZxLeWEmyMDpHSuvFqnzY3buoQ3wa/G1Xl4OPd |
| | UvzX5zdTSPo4rpCXB1QkN6QIDAQAB |
| Decrypted information | MFG: Coltene |
| (Cipher: RSA/ECB/PKCS1Padding, | TYPE: Class 5 |
| plain text display) | MFGDATE: YYYYMMDD |
| | BATCH: YYWWBBBBBB |
| | SN: AA000000A00001 |
| | CHK: XXXXXXXXXXXXXXXX |

FIG. 4B illustrates an example of a 2D code representation of the Encrypted Class 5 indicator information.

A class 4 indicator ID may be similarly encrypted or decrypted as a class 5 indicator ID as provided in the example in Table 1 above. Class 4 or 5 indicator ID may include identification of the class 4 or 5 indicator, time stamps and serial number information. As illustrated in FIG. 5, the Indicator ID (without encryption) of a class 4 or 5 indicator and related status, such as unused, may be stored, by the server 120, into a block n of a blockchain in a remote server 120, together with time stamp of creation of the class 4 or 5 indicator. In the example of FIG. 5, the structure of block n includes a timestamp to indicate of time of generating the indicator ID, a hash value for block n−1, a hash value for block n, an indicator ID (unencrypted), and the status of the indicator as used. The structure of block n may also include additional information, such as information related to wash step and treatment step. In some examples, the wash step and treatment step of an instrument may be recorded, by the server 120, in blocks of separate blockchains. The Hash value for block n, namely Hash n, is calculated by applying hash function over Timestamp, Indicator ID and Status of block n, and previous Hash value in block n−1, namely Hash n−1. The hash function may be MD5, RIPEMD, SHA-1, and SHA-256. A one-way hash function maps data of arbitrary size (called the “message”) to a bit string of a fixed size (the “hash value”, “hash”, or “message digest”). Each of hash for block [n−1], and hash for block [n] generates a message digest. The blockchain information can be later used to enquire about ID status and origin time stamp.

In an example, for Block 0, the hash of the previous block [n−1] may be set to 0, while the hash for current block, Hash n, may be is calculated over the content of Timestamp, Indicator ID, Status, Sterilization log and previous Hash fields. For block 1, the previous hash field Hash [n−1] is initialized to 0 calculated for block 0. Current hash for block 1 is calculated over the content of Timestamp, Indicator ID, Status, Sterilization log and previous Hash field Hash [n−1]. For block n, the previous hash filed is initialized to the hash value as calculated for Block n−1. Current hash for block n is calculated over the content of Timestamp, Indicator ID, Status, Sterilization log and previous Hash field Hash [n−1].

In FIG. 1, with respect to a wrapped cassette 101 having a successful sterilization cycle, the cassette ID and the Load ID associated with the cassette 101 may be scanned prior to being stored, for example, in a blockchain at the server 120. After the cassette 101 is unwrapped, the class 4 chemical indicator is accessible to a user. With a successful sterilization, the class 4 indicator is revealed and becomes readable to indicate that instruments in the cassette 101 are successfully sterilized. The sterilized instruments in the cassette 101 may be allocated to a patient for treatment. Once instruments are required for procedures, the Load ID may be scanned and linked to a patient file, and thus provides a permanent link and traceability to the reprocessing cycle of the instruments. In this case, the cassette ID, the load ID, the class 4 or class 5 chemical indicator ID, and each instrument in the cassette 101 are associated

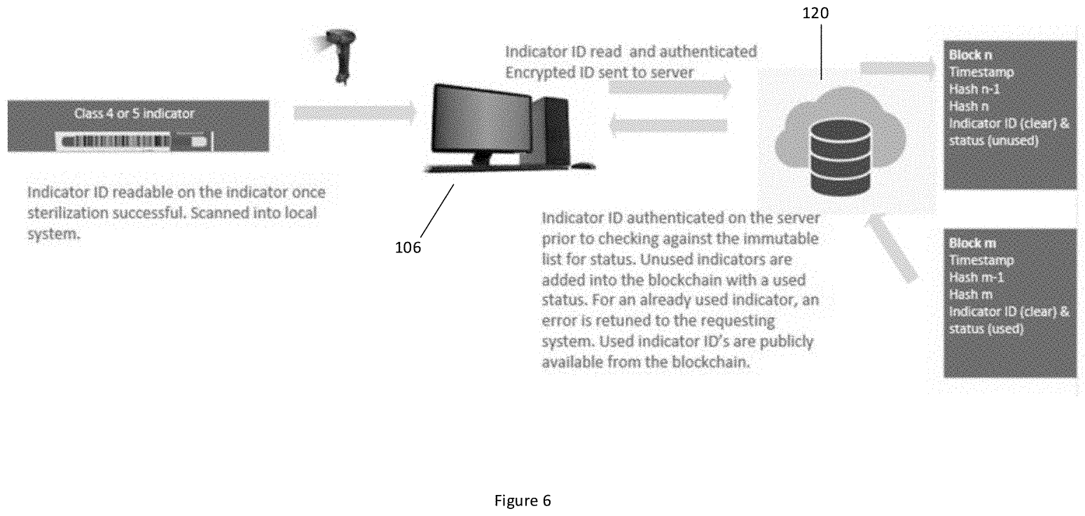

As illustrated in FIG. 6 and described above, upon exposure to a successful sterilization cycle at the sterilization equipment 104, the encrypted class 5 indicator ID is revealed and becomes readable. When the class 5 indicator ID is scanned in the local computer 106 or sterilizer 104, the class 5 indicator ID can be authenticated, at the server 120 or computer 106, by using a predetermined public server key, such as a Coltene public key.

After the class 5 indicator ID is authenticated at the server 120 or computer 106, the local computer 106 or sterilizer 104 may send the encrypted class 5 indicator ID to the remote server 120. The remote server 120 or the computer 106 may use a public key from the trusted third party to decrypt the ID. The remote server 120 may compare the decrypted ID with the indicator ID store in the block n against the immutable blockchain record when the indicator ID is initially created to validate the decrypted ID. In some examples, the remote server 120 may create a new block, such as block n in FIG. 6, to record the completion of the sterilization of the instruments in the cassette 101 as indicated in the decrypted indicator ID.

To simplify the process, the original encrypted information with a private key may be sent to the remote server 120 for authentication. With encrypted information, the server 120 can authenticate the encrypted information using a public key to decrypt the encrypted information. After the indicator ID is authenticated, when an instrument associated with the indicator ID is used in a procedure for treatment, the server 120 may update the blockchain related to the instrument by adding a new block with a new block m, which comprises a timestamp when the block m is generated, hash m−1, hash m, unencrypted indicator ID, and a status set as used. A confirmation may be sent back to the local computer 106 regarding the change of status of the indicator to used. An error may be reported, by the server 120, to the local office computer 106 if the record of the indicator ID is not found in the blockchain or if the status of the block with the indicator ID is found to be marked as used. The authentication mechanism and the blockchain therefore may protect against malicious actors trying to abuse the reprocessing tracing system.

If a load has an unsuccessful sterilization cycle, the code of class 4 or 5 medical indicator is not revealed and thus cannot be scanned, therefore the load with the Load ID cannot be safely released, and the instruments in the cassette 101 require repackaging and reprocessing at least for the sterilization step.

The system 100 uses reprocessing equipment 102 and 104 and associates each of the cassette ID, the load ID, and the class 4 or chemical indicator code with each instrument in the cassette 101. This allows tracking of instruments in the cassette 101 throughout the infection reprocessing process. As such, a specific instrument used in treatment of a patient or a patient file is associated with a specific cassette 101, a specific washing cycle, and a specific sterilization step. As well, a machine readable chemical indicator (class 4 and 5) becomes readable only on sterilization process success.

As well, the revealed code of class 4 or 5 chemical indicator may be read and automatically authenticated through the use of asymmetric encryption by using public key and private key, and finally use of blockchain technology to document and file chemical indicator (CI) and biological indicator (BI) to successful sterilization cycles with trusted third parties.

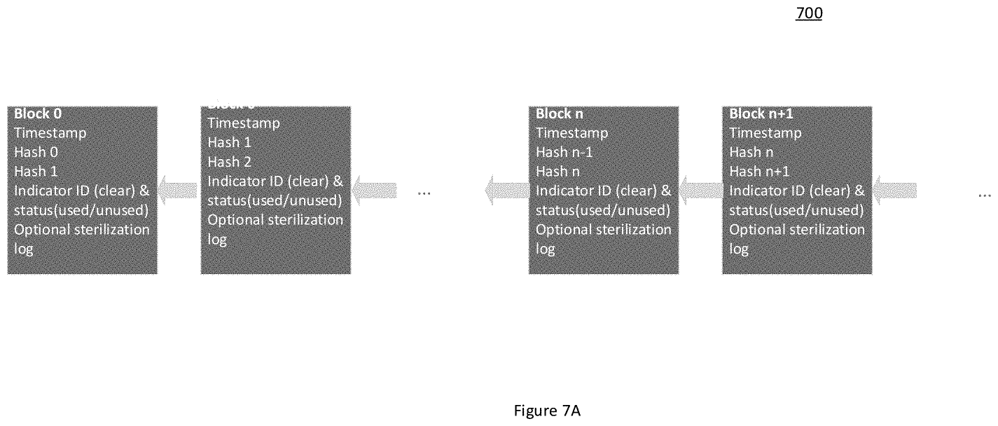

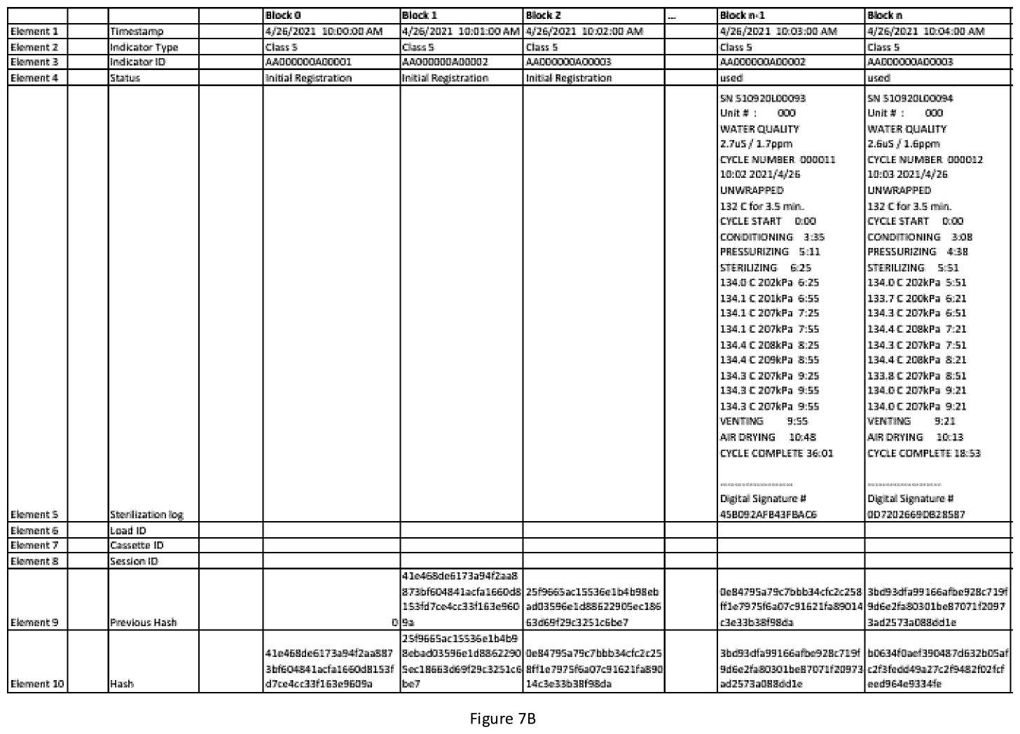

As illustrated in FIG. 7A, a block chain 700 may include a plurality of blocks. Each block are interconnected with a hash function of the immediate previous block. The block may also include other information, such as one or more of indicator type, sterilization log, load ID, cassette ID, session ID. Session ID represents a set of infection control operations that are applied to the instruments in a cassette 101 from dirty to clean, for example a wash in the ultrasonic washer, a disinfection cycle and finally a sterilization cycle. A new session ID is generated by the computer 106 once dirty instruments are returned for reprocessing. Each block may include a sterilization log, which may include information related to sterilization, such as the Sterilizer model and software version, serial number of the sterilizer, sterilization cycle type and sterilization parameters, timed stamped sterilization phases. Status for the sterilization end condition may also be added to the sterilization log. For example, a sterilization log may include the following information:

STATIM 5000 S503R751
SN 510920L00093
Unit #: 000
WATER QUALITY
2.7 uS/1.7 ppm
CYCLE NUMBER 000011
8:02 18 Dec, 2020
UNWRAPPED
132 C for 3.5 min.
CYCLE START 0:00
CONDITIONING 3:35
PRESSURIZING 5:11
STERILIZING 6:25
134.0 C 202kPa 6:25
134.1 C 201kPa 6:55
134.1 C 207kPa 7:25
134.1 C 207kPa 7:55
134.4 C 208kPa 8:25
134.4 C 209kPa 8:55
134.3 C 207kPa 9:25
134.3 C 207kPa 9:55
134.3 C 207kPa 9:55
VENTING 9:55
AIR DRYING 10:48
CYCLE COMPLETE 36:01
Digital Signature #
45B092AFB43FBAC6

Pairing of the private or public key with the blockchain allows for the records to be authenticated as well as identifying if successfully used and when. The blockchain 700 may store, in the server 120, in each of the blocks class 4 and/or 5 chemical indicator ID, indicator Class, time stamp and optionally the sterilization log for successful cycles. The instruments in a cassette 101 have the same class 4 or 5 indicator ID, same load ID and cassette ID. FIG. 7B provides another exemplary block structure. In FIG. 7B, a block may include a timestamp, an indicator type to indicate a class 4 or class 5 indicator, an indicator ID to indicate the ID of the class 4 or 5 indicator, status of the instrument such as “initial registration” or used), a sterilization log to include information related to sterilization, a load ID to identify specific load, a cassette ID to identify a specific cassette, a session ID to identify a specific reprocessing step, hash value of the previous block, and hash value of the current block.

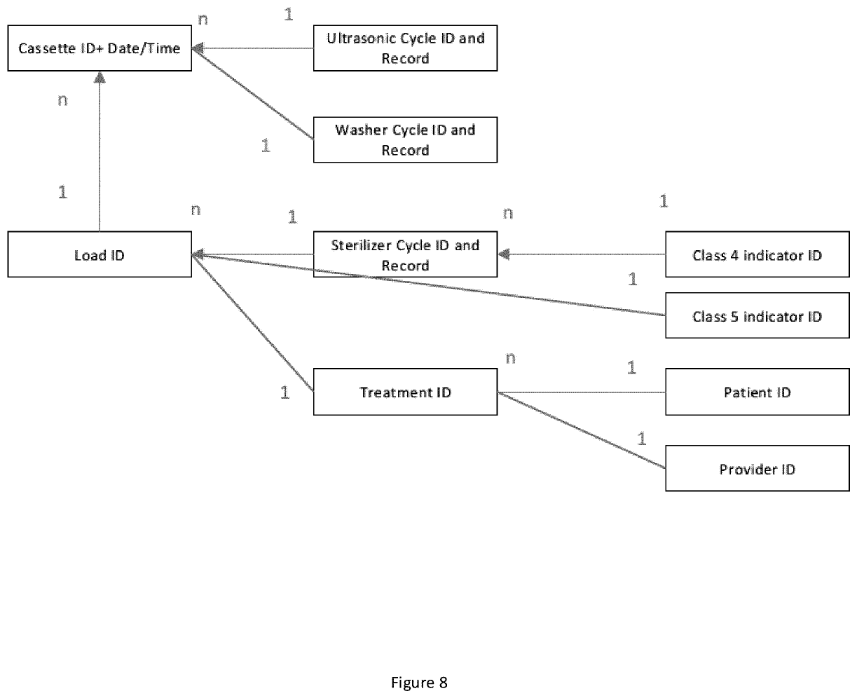

As illustrated in the example of FIG. 8, an original cassette ID may include additional information in a desired format, such as date and time information, to create a new uniquely identifiable identification (new ID) of the cassette. Accordingly, one or more instruments in the cassette are also associated with the new ID. The additional information may form a portion of the new identification, for example, by appending to the end of original cassette ID. The new ID may be used to track one or more instruments in the cassette in a reprocessing process and in the blockchain described above. For example, the cassette ID described above may be the new ID.

As well, the new ID can be linked to or associated with an ultrasonic cycle ID and/or a washer cycle ID prior to washing of one or more instruments. The new ID may be associated with both ultrasonic cycle ID and the washer cycle ID and therefore is a “1” to “n” relationship with the ultrasonic cycle ID and the washer cycle ID. The ultrasonic cycle ID is a unique identification for an ultrasonic cleaning cycle of a washer. The washer cycle ID is a unique identification for a washing cycle of a washer. Linking or associating the new ID with the ultrasonic cycle ID or washer cycle ID indicates that the one or more instruments in the cassette are washed at an ultrasonic cleaning cycle or a washing cycle of a washer with the ultrasonic cycle ID or washer cycle ID. As such, the ultrasonic cycle ID also indicates an ultrasonic cycle or wash record of the one or more instruments in a cassette at a specific ultrasonic cycle of an ultrasonic cleaning cycle or a washing cycle of a washer. As such, the new ID may also reflect the ultrasonic washing cycle or a washer cycle.

As well, a class 4 and/or 5 indicator may be associated with a unique sterilization ID and record. The sterilization ID may be associated with both class 4 indicator ID and the class 5 indicator ID and therefore is a “1” to “n” relationship with the class 4 indicator ID and the class 5 indicator ID. As described above, a lass 4 and/or 5 indicator is revealed for reading or scanning only after a sterilization of instruments in a cassette is successfully performed. The sterilization ID may include information of specific sterilization equipment and sterilization cycle information. Association of a class 4 and/or 5 indicator with the unique sterilization ID indicates that the one or more instruments in the cassette are successfully sterilized at a specific sterilization cycle of a sterilization equipment. As such, the sterilization ID also indicate a sterilization record of the one or more instruments in a cassette at a specific sterilization cycle of a sterilization equipment.

After a unique sterilization ID and record is associated with a class 4 and/or 5 indicator, the sterilization ID may be associated with a unique Load ID of an instrument.

As such, the Load ID is associated with an ultrasonic cycle or wash record of the instrument a sterilization record of the instrument, and a class 4 and 5 indicator of the instrument. The Load ID indicates a full reprocessing cycle of an instrument.

As well, the Load ID may be associated with a treatment ID with which the instrument associated with the Load ID may be used to treat a patient. As illustrated in FIG. 8, the load ID may be associated with both the sterilization ID and treatment ID therefore is a “1” to “n” relationship with the sterilization ID and treatment ID.

As illustrated in FIG. 8, the instrument is provided by a provider or health practitioner, which is associated with a provider ID. When the instrument is used to treat a patient, the patient has a patient ID. The treatment ID may be associated with both provider ID and patient ID therefore is a “1” to “n” relationship with the provider ID and patient ID. The instrument is used in a treatment, which is associated with a treatment ID to indicate the treatment. By with the instrument used in the treatment of the patient, the treatment ID is associated with the provider ID and the patient ID. The Load ID of the instrument can be associated with the treatment ID. As such, the instrument used to treat a patient may be traced back to each reprocessing step of the instrument.

Certain adaptations and modifications of the described embodiments can be made. Therefore, the above discussed embodiments are considered to be illustrative and not restrictive.

1. A method for tracking a cassette housing one or more instruments in an instrument reprocessing, comprising:
receiving, at a server, a chemical indicator identification encrypted by a private key after the cassette is sterilized;
decrypting, at the server, the chemical indicator identification with a corresponding public key; and
in response to decryption of the chemical indicator identification, authenticating, at the server, the chemical indicator identification.

2. The method of claim 1, wherein the chemical indicator identification comprises a class 4 chemical indicator identification.

3. The method of claim 1, wherein the chemical indicator identification comprises a class 5 chemical indicator identification.

4. The method of claim 1, wherein the chemical indicator identification comprises both a class 4 chemical indicator identification and a class 5 chemical indicator identification.

5. The method of claim 1, further comprising storing the decrypted chemical indicator identification in a block of a blockchain and set a status of the cassette as unused.

6. The method of claim 5, further comprising, after the one or more instruments in the cassette are used, adding a new block to the blockchain and modifying the status of the cassette as used.

7. The method of claim 5, wherein the block comprises a timestamp indicating a time of generating the block, the chemical indicator identification, a cassette identification, a previous block hash value, and a current block hash value.

8. The method of claim 5, wherein the block further comprises a sterilization log comprising data related to sterilization of the cassette.

9. The method of claim 8, wherein the block further comprises one or more of an indicator type for indicating a type of the chemical indicator, a load identification, and a session identification for indicating a reprocessing step.

10. The method of claim 7, wherein the cassette identification comprises date and time information when the cassette is reprocessed.
